(19) United States
(12) Patent Application Publication
(10) Pub. No.: US 2012/0176584 A1
(43) Pub. Date: Jul. 12, 2012

(54) Hand-Held Medical Device For Illumination

(75) Inventors: Volker Geuder, Heidelberg (DE); René Draheim, Sandhausen (DE)

(73) Assignee: Geuder AG, Heidelberg (DE)

(21) Appl. No.: 13/379,904

(22) PCT Filed: Jun. 21, 2010
(86) PCT No.: PCT/DE2010/000697

§ 371 Date: Mar. 26, 2012

(30) Foreign Application Priority Data
Jun. 22, 2009 (DE) 10 2009 030 060.0

(51) Int. Cl. A61B 3/10

(52) U.S. Cl. 351/221

(57) ABSTRACT

The invention relates to a hand-held medical device for illumination, in particular for use in ophthalmology, comprising a housing used as a handle piece, a tube (1), which extends out of the housing and has an optical waveguide (2) running through it and a light outlet at the end of the tube, and a light source (3) integrated in the hand-held device, the hand-held medical device being characterized in that the light source (3) is an LED light source having a surface (4) that emits light and that the optical waveguide (2) directly adjoins the surface (4) that emits light.

The invention relates to a medical handheld device for lighting, especially for use in ophthalmology, with a housing that serves as a handle, a probe extending from the housing with a fiber optic running through it and a light outlet at the end, and a light source integrated in the housing.

Medical handheld devices for lighting are known in various embodiments in practice. In the case of handheld devices for use in ophthalmology, generally a small construction is necessary. This applies especially to the probe extending from the housing that has a light outlet on the end. If the probe is used for introduction into the human body, for example into the human eye, in addition to the small construction, unrestricted handling of the instrument is also necessary.

Category-defining medical handheld devices of this type, especially for use in ophthalmology, are known e.g., from DE 10 2006 051 736 A. They have an extremely small construction and comprise an LED light source in the handle. Such manual devices are supplied with power either from a battery located in the handle or by way of a power connection leading outward that is generally connected to a control unit.

Even though LEDs currently have considerable light intensity, this is far from comparable to conventional lamps, e.g., halogen lamps. If the LED is used for coupling light into a fiber optic, e.g. into a glass fiber, considerable losses occur, especially since the LED causes extreme scattering of the light and only a small part of the light emitted is actually available for coupling to the glass fiber. This makes the use of LEDs in generic medical manual devices problematic, even though the miniaturization involved brings many advantages.

The present invention is thus based on the object of designing and further developing a medical manual device for lighting, especially for use in ophthalmology, in such a way that with the use of a suitable LED light source, an optimal light yield and thus an adequately good illumination is possible using a probe comprising a fiber optic.

According to the invention, the object above is achieved by a medical manual device with the characteristics of claim 1. According to it, the category-defining medical manual device is characterized in that the light source is defined as an LED light source with a light-emitting surface and that the fiber optic is directly adjacent the light-emitting surface.

According to the invention, it has been recognized that for coupling to a fiber optic, LED light sources with a light-emitting surface are especially suitable if the fiber optic is not directly adjacent to the light-emitting surface. In this case, LEDs with so-called bonding wires in an otherwise usual transparent covering element are not used. Rather, this involves the use of LED light sources that do not comprise a light-emitting surface that are coated in the usual way with a type of silicon lens and/or with a covering mask. Such LED light sources scatter the light almost 180°.

In the manner of the invention, it has also been recognized that such an LED light source—without the cover element—can be used in an ideal way for coupling light in a fiber optic, namely if the fiber optic is directly adjacent to the light-emitting surface, namely if the fiber optic is placed flush on the surface. The light is thus not scattered through the otherwise usual lens, rather it goes directly from the light-emitting surface into the fiber optic so a maximum/optimal light yield is achieved.

In an advantageous manner, the LED light source is a so-called light chip, as is known from DE 20 2006 018 846—considered by itself. What is important there is that in the light chip, a light-emitting surface is provided that—with appropriate structure of the light chip—can be designed as a round surface, corresponding to the cross section of the fiber optic. Adaptation of the light-emitting surface to the cross-section surface of the fiber optic is advantageous.

The fiber optic can be a classic fiber optic, preferably a glass fiber that extends from the handle of the medical manual device, through the probe, to the open end of the probe. Light is emitted there.

Concretely, the fiber optic is a monofilament or a fiber bundle, as required. The probe containing the fiber optics can be straight or also bent. This is considered a special advantage of fiber optics, in that almost any design of the probe is conceivable.

For optimal coupling of the light in the fiber optic, it is also advantageous if the fiber optic is aligned approximately orthogonally to the light-emitting surface. In this case, the fiber optic can be adjacent to the light-emitting surface with at least one flat surface or can contact this surface consistently without a gap remaining between them. In principle, it is conceivable for the fiber optic to be held in its position with respect to the light-emitting surface using appropriate holding/positioning/calibrating means.

In the scope of an especially simple design of positioning the fiber optic to the light-emitting surface of the LED light source, it is advantageous if the fiber optic—on the end—is glued to the light-emitting surface, preferably flush. The glue involves a transparent glue that is resistant to temperature and aging in the area of the heat that develops there. An adaptation of thermal expansion coefficients of the different materials is also advantageous.

Concretely, it is conceivable that the glue covers the light-emitting surface completely in the sense of a covering mask—instead of the usual lens element—whereby the fiber optic dips into the glue for contact with the light-emitting surface, and in fact, preferably until there is full contact with the light-emitting surface.

As already mentioned, it is advantageous if the light emitted from the surface is coupled completely in the fiber optic. It is advantageous if the area extending over the cross-section surface of the fiber optic is covered by an impermeable cover mask so the light emission is concentrated on the cross section surface of the fiber optic. Alternatively, and/or additionally, the light-emitting surface can be adapted to the fiber cross section so that further design measures are unnecessary.

Finally, it can also be advantageous if the connecting area between the fiber optic and the light-emitting surface is sealed optically and/or mechanically. A mechanical sealing is understood to mean a stabilizing of the connection. Optical sealing is used to wall off the light source toward the outside so that all of the light is available for coupling in the fiber optic. The sealing can be produced in the sense of a covering, which covers the light source completely and the end area of the fiber optic and thus also promotes and/or stabilizes the connection between the LED light source and the fiber optic.

There are now various possibilities for designing and further developing the teaching of the present invention in an advantageous manner. On one hand, reference is made to the claims subordinate to claim 1 and on the other, to the following explanation of an exemplary embodiment of the invention using the drawing. In connection with the explanation of the preferred exemplary embodiment of the invention using the drawings, preferred designs and further developments are explained generally in the teaching. In the drawings

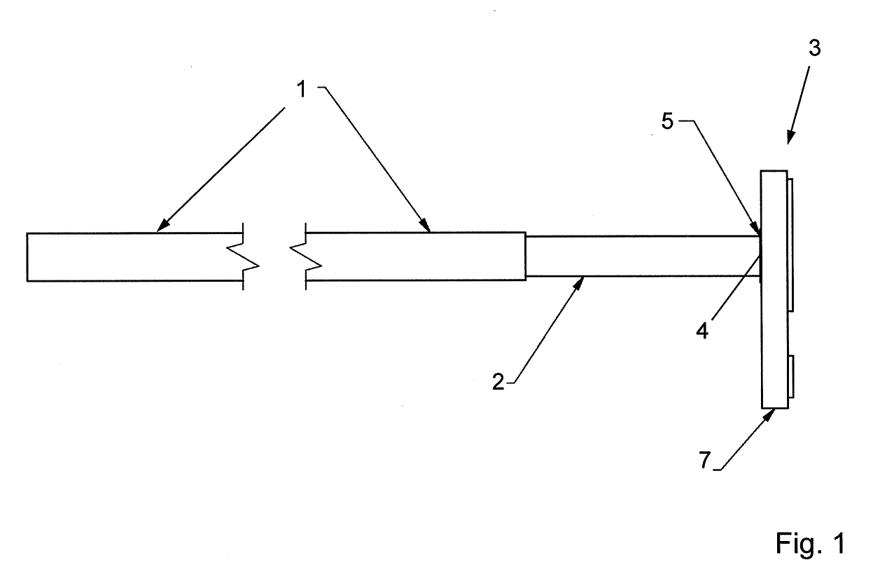

FIG. 1 shows a schematic view of an exemplary embodiment of a light source with light coupling to a fiber optic for use in a medical manual device according to the invention and

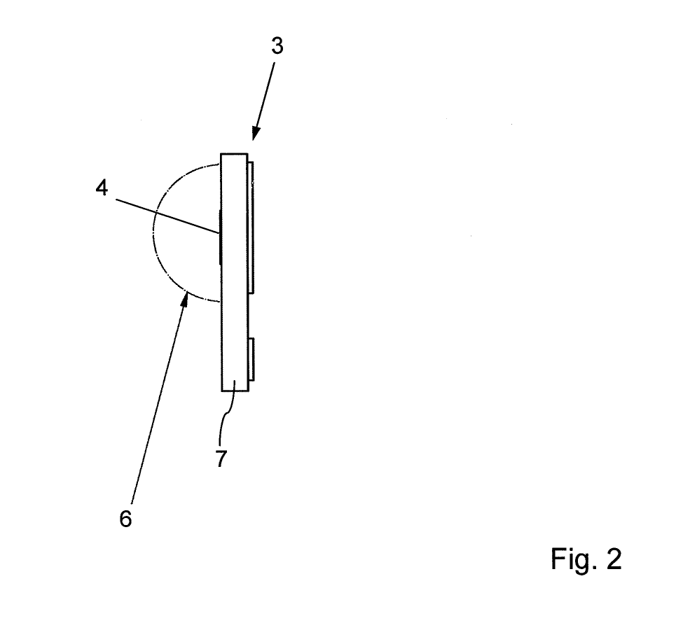

FIG. 2 shows the LED light source used in the conventional form.

FIG. 1 shows a part of the “inner life” of a medical handheld device for lighting, especially for use in ophthalmology, where such a manual device comprises a housing not shown in the figures, which serves in the usual way as a handle. From the housing, a probe 1 extends with a fiber optic 2 running in it. The fiber optic 2 is, for example, but not necessarily, a monofilament of glass.

In addition, an LED light source 3 integrated in the housing that is not shown is provided, the light of which is coupled in the fiber optic 2. For the sake of simpler representation, the electrical connections of the LED light source and the power supply are not shown, especially since the claimed teaching does not deal with them.

In principle, the fiber optic 2 can also be a fiber bundle. If monofilaments are used, in ophthalmology, fiber cross sections from 20 to 25 gauge are advantageous.

FIG. 1 also shows that the LED light source 3 has a light-emitting surface 4. In the area of the light-emitting surface 4, the fiber optic 2, or more specifically the monofilament, is coupled to the light-emitting surface so the light emitted by the LED light source 3 can be fed directly into the fiber optic 2.

The connection between the LED light source 3 and/or the light-emitting surface 4 and the fiber optic 2 is produced by a transparent adhesive 5.

FIG. 2 shows the LED light source 3 used in FIG. 1 in a condition that is usual on the market, according to which the light-emitting surface 4 is covered by a transparent lens 6 called a cover mask. Depending on the design of the lens, the light is emitted in a range of almost 180° to the substrate 7 and/or carrier of the LED light source 3.

Finally it should be noted that the exemplary embodiment explained above is only the explanation of the medical manual device according to the invention, but this is not restricted to the exemplary embodiment.

### REFERENCE NUMBER LIST

1 Probe
2 Fiber optic
3 LED light source
4 Light-emitting surface (of 3)
5 Adhesive
6 Lens
7 Substrate

1-13. (canceled)

14. A medical manual device for lighting, especially for use in ophthalmology, said medical manual device comprising:
a housing serving as a handle;
a probe extending from the housing with a fiber optic running in it and a light outlet at the end; and
a light source integrated in the housing,
wherein the light source is designed as an LED light source with a light-emitting surface, and wherein the fiber optic is directly adjacent to the light-emitting surface.)

15. The medical manual device according to claim 14, wherein the light source is designed as a light chip.

16. The medical manual device according to claim 14, wherein the fiber optic is designed as a fiber optic, preferably as a glass fiber.

17. The medical manual device according to claim 16, wherein the fiber optic is designed as a monofilament.

18. The medical manual device according to claim 16, wherein the fiber optic is designed as a fiber bundle.

19. The medical manual device according to claim 14, wherein the fiber optic is aligned approximately orthogonally to the light-emitting surface.

20. The medical manual device according to claim 14, wherein the fiber optic is adjacent to the light-emitting surface with at least one plane surface.

21. The medical manual device according to claim 14, wherein the fiber optic—at the end—is glued to the light-emitting surface, preferably flush.

22. The medical manual device according to claim 21, wherein the fiber optic is glued to the light-emitting surface by means of transparent glue.

23. The medical manual device according to claim 22, wherein the glue covers the light-emitting surface completely in the manner of a cover mask—instead of a lens element.

24. The medical manual device according to claim 14, wherein the area of the cover mask extending over the cross-section surface of the fiber optic is at least largely impermeable to light.

25. The medical manual device according to claim 21, wherein the light-emitting surface is somewhat adapted to the fiber cross section.

26. The medical manual device according to claim 14, wherein the connecting area between fiber optic and light-emitting surface is optically and/or mechanically sealed.
